SEQUENCE LISTING (54 sequences)

SEQ ID NO: 1 - protein, 17 aa, Artificial Sequence
  Recombinant synthetic protein
HKCAKIKWRGVHVKYCA

SEQ ID NO: 2 - protein, 17 aa, Artificial Sequence
  Recombinant synthetic protein
KGCALVKVRGLTLKVCK

SEQ ID NO: 3 - protein, 17 aa, Artificial Sequence
  Recombinant synthetic protein
KWCRKWQWRGVKFIKCV

SEQ ID NO: 4 - protein, 17 aa, Artificial Sequence
  Recombinant synthetic protein
AICRTWKYRGHKWKACK

SEQ ID NO: 5 - protein, 17 aa, Artificial Sequence
  Recombinant synthetic protein
RKCFPYRFRGKRFKKCY

SEQ ID NO: 6 - protein, 17 aa, Artificial Sequence
  Recombinant synthetic protein
KKCFTWKWRGKNYRKCG

SEQ ID NO: 7 - protein, 17 aa, Artificial Sequence
  Recombinant synthetic protein
RGCWRWKWRGVWYKKCL

SEQ ID NO: 8 - protein, 17 aa, Artificial Sequence
  Recombinant synthetic protein
GKCWKWKYRGHYWRTCL

SEQ ID NO: 9 - protein, 17 aa, Artificial Sequence
  Recombinant synthetic protein
KNCLKWKWRGITYRKCL

SEQ ID NO: 10 - protein, 17 aa, Artificial Sequence
  Recombinant synthetic protein
IRCATWNYRGHQWKKCL

SEQ ID NO: 11 - protein, 17 aa, Artificial Sequence
  Recombinant synthetic protein
GNCKVFQYRGKRWARCL

SEQ ID NO: 12 - protein, 17 aa, Artificial Sequence
  Recombinant synthetic protein
LGCKRFKFRGITWKGCI

SEQ ID NO: 13 - protein, 17 aa, Artificial Sequence
  Recombinant synthetic protein
LTCRKWYYRGVHWKVCV

SEQ ID NO: 14 - protein, 17 aa, Artificial Sequence
  Recombinant synthetic protein
ANCKIWNWRGKRYKKCV

SEQ ID NO: 15 - protein, 17 aa, Artificial Sequence
  Recombinant synthetic protein
IGCLRWRYRGVTWRKCV

SEQ ID NO: 16 - protein, 17 aa, Artificial Sequence
  Recombinant synthetic protein
KNCIKYHYRGINYRSCG

SEQ ID NO: 17 - protein, 17 aa, Artificial Sequence
  Recombinant synthetic protein
KKCGAFTYRGVHYRKCV

SEQ ID NO: 18 - protein, 17 aa, Artificial Sequence
  Recombinant synthetic protein
AVCGKFHWRGVKYRICK

SEQ ID NO: 19 - protein, 17 aa, Artificial Sequence
  Recombinant synthetic protein
AKCKSFYYRGKWFGKCY

SEQ ID NO: 20 - protein, 17 aa, Artificial Sequence
  Recombinant synthetic protein
KWCRVFHYRGNKWKLCY

SEQ ID NO: 21 - protein, 17 aa, Artificial Sequence
  Recombinant synthetic protein
AWCGAWRYRGKHYIKCR

SEQ ID NO: 22 - protein, 17 aa, Artificial Sequence
  Recombinant synthetic protein
RNCLKWTWRGITYLKCK

SEQ ID NO: 23 - protein, 17 aa, Artificial Sequence
  Recombinant synthetic protein
AICRAYKYRGHKWGICA

SEQ ID NO: 24 - protein, 17 aa, Artificial Sequence
  Recombinant synthetic protein
KKCALWKFRGHKWVRCA

SEQ ID NO: 25 - protein, 17 aa, Artificial Sequence
  Recombinant synthetic protein
LLCLKWKYRGHTYRGCL

SEQ ID NO: 26 - protein, 17 aa, Artificial Sequence
  Recombinant synthetic protein
LKCIGWTYRGHKWRSCF

SEQ ID NO: 27 - protein, 17 aa, Artificial Sequence
  Recombinant synthetic protein
KKCKGYWWRGVRYKICK

SEQ ID NO: 28 - protein, 17 aa, Artificial Sequence
  Recombinant synthetic protein
AGCKIYRWRGHTWKICG

SEQ ID NO: 29 - protein, 17 aa, Artificial Sequence
  Recombinant synthetic protein
FKCGAWHYRGNRWVKCL

SEQ ID NO: 30 - protein, 17 aa, Artificial Sequence
  Recombinant synthetic protein
IWCLRWHYRGKKYAVCA

SEQ ID NO: 31 - protein, 17 aa, Artificial Sequence
  Recombinant synthetic protein
IRCKKYSFRGVHYVSCA

SEQ ID NO: 32 - protein, 17 aa, Artificial Sequence
  Recombinant synthetic protein
GNCKGYHWRGHKFKLCL

SEQ ID NO: 33 - protein, 17 aa, Artificial Sequence
  Recombinant synthetic protein
FLCKSWKWRGKYYIRCL

SEQ ID NO: 34 - protein, 17 aa, Artificial Sequence
  Recombinant synthetic protein
GLCRLYKYRGVKYKSCL

SEQ ID NO: 35 - protein, 17 aa, Artificial Sequence
  Recombinant synthetic protein
KWCIKFSYRGIWWKACR

SEQ ID NO: 36 - protein, 17 aa, Artificial Sequence
  Recombinant synthetic protein
LKCGTWRFRGHKWKVCY

SEQ ID NO: 37 - protein, 17 aa, Artificial Sequence
  Recombinant synthetic protein
LKCKVYRFRGIRYGKCL

SEQ ID NO: 38 - protein, 17 aa, Artificial Sequence
  Recombinant synthetic protein
AFCAKYRFRGKRYVGCA

SEQ ID NO: 39 - protein, 17 aa, Artificial Sequence
  Recombinant synthetic protein
KNCFKWTYRGHYWKSCA

SEQ ID NO: 40 - protein, 17 aa, Artificial Sequence
  Recombinant synthetic protein
KLCKLYTYRGKYWGKCK

SEQ ID NO: 41 - protein, 17 aa, Artificial Sequence
  Recombinant synthetic protein
AKCARYNYRGKKWLKCR

SEQ ID NO: 42 - protein, 17 aa, Artificial Sequence
  Recombinant synthetic protein
FGCRKFYWRGVKWKVCA

SEQ ID NO: 43 - protein, 17 aa, Artificial Sequence
  Recombinant synthetic protein
GICKTWNYRGKKYKICL

SEQ ID NO: 44 - protein, 17 aa, Artificial Sequence
  Recombinant synthetic protein
RNCAFWKWRGKSYALCK

SEQ ID NO: 45 - protein, 17 aa, Artificial Sequence
  Recombinant synthetic protein
KICAKYNWRGKTYKICL

SEQ ID NO: 46 - protein, 17 aa, Artificial Sequence
  Recombinant synthetic protein
AICARWKWRGISYKRCR

SEQ ID NO: 47 - protein, 17 aa, Artificial Sequence
  Recombinant synthetic protein
IFCWGYKFRGVHYKACR

SEQ ID NO: 48 - protein, 17 aa, Artificial Sequence
  Recombinant synthetic protein
KTCAKWSYRGVNYGRCR

SEQ ID NO: 49 - protein, 17 aa, Artificial Sequence
  Recombinant synthetic protein
AKCSVYTWRGNKWRTCK

SEQ ID NO: 50 - protein, 17 aa, Artificial Sequence
  Recombinant synthetic protein
IRCAVWKYRGNKYKTCA

SEQ ID NO: 51 - protein, 17 aa, Artificial Sequence
  Recombinant synthetic protein
KLCKTWQWRGHTWRTCI

SEQ ID NO: 52 - protein, 17 aa, Artificial Sequence
  Recombinant synthetic protein
KICGKYHFRGVQYKACK

SEQ ID NO: 53 - protein, 17 aa, Artificial Sequence
  Recombinant synthetic protein
KKCKAYTFRGVYWKACL

SEQ ID NO: 54 - protein, 17 aa, Artificial Sequence
  Recombinant synthetic protein
LKCRTWNWRGKKYALCK

(19) United States
(12) Patent Application Publication
(10) Pub. No.: US 2017/0128390 A1
(43) Pub. Date: May 11, 2017

(54) DISINFECTANT AND ANTIMICROBIAL COMPOSITIONS, IN PARTICULAR FOR THE VETERINARY FIELD

(71) Applicant: I.C.F. S.R.L., Palazzo Pignano (CR) (IT)

(72) Inventors: Clotilde Silvia CABASSI, Parma (PR) (IT); Gennaro FALANGA, Gerre De' Caprioli (CR) (IT); Antonello ROMANI, Parma (PR) (IT)

(21) Appl. No.: 15/318,570

(22) PCT Filed: Jun. 25, 2015
(86) PCT No.: PCT/IB2015/054790

§ 371 Date: Dec. 13, 2016

(30) Foreign Application Priority Data
Jun. 27, 2014 (IT) MI2014A001176

(51) Int. Cl. A61K 31/155; A61K 47/18; A61K 38/16; A61Q 5/02; A61K 38/40; A61K 38/17; A61K 8/41; A61K 8/64; A61K 8/43; A61Q 17/00; A61K 9/06; A61K 9/08; A61K 38/10; A61Q 19/10; A61L 2/18; A61K 9/00

(52) U.S. Cl. CPC A61K 31/155; A61K 9/0014; A61K 9/0046; A61K 47/18; A61K 47/183; A61K 38/16; A61Q 5/02; A61K 38/40; A61K 38/1729; A61K 8/41; A61K 8/64; A61K 8/43; A61Q 17/005; A61K 9/06; A61K 9/08; A61K 38/10; A61Q 19/10; A61L 2/18

(57) ABSTRACT

There are described compositions comprising chlorhexidine or a salt thereof and at least one peptide, and their use in the treatment of infections caused by bacteria, fungi and/or yeasts, in particular in the veterinary field.

### FIELD OF THE INVENTION

The present invention relates to compositions comprising chlorhexidine or a salt thereof, and at least one peptide, for use in the treatment of infections caused by bacteria, fungi and/or yeasts, in particular in the veterinary field.

### BACKGROUND ART

Chlorhexidine is a cationic polybiguanide (bisbiguanide). It is used mainly in the form of salt, for example dihydrochloride, diacetate and digluconate.

As a biocide, its target is the bacterial cell wall. At low concentrations, chlorhexidine binds to the negatively charged cell wall and disrupts the osmotic balance. At higher concentrations, chlorhexidine attacks the bacterial cytoplasmic membrane and denatures the microbial proteins. Chlorhexidine has both a rapid onset of bactericidal action and a prolonged antimicrobial efficacy through residual effects.

Depending on the formulation and concentration, chlorhexidine is effective as bactericide, virucide and fungicide.

In particular, at low concentrations, chlorhexidine is effective against most gram-positive bacteria.

At higher concentrations, chlorhexidine is effective against gram-negative bacteria. At the highest concentrations, chlorhexidine is effective against yeasts.

The virucidal activity is good against ‘coated’ viruses (such as HIV, cytomegalovirus, influenza, respiratory syncytial virus and herpes virus), but not against ‘naked’ viruses (such as rotaviruses, adenoviruses and enteroviruses).

Chlorhexidine has no sporicidal activity, therefore it is not effective against Clostridium difficile spores and it is not active against mycobacteria.

Thanks to its broad activity spectrum, its acceptable tolerability and a good level of safety, chlorhexidine is one of the most frequently used antiseptic agents. The reduced availability of triclosan products following concerns about the safety and the selection of the antimicrobial resistance has exacerbated the growing exposure to chlorhexidine. In addition, there is a growing emphasis on the control of the methicillin-sensitive S. aureus (MSSA), which probably increases the further use of products containing chlorhexidine.

In a recent publication by Homer C. et al. (“Reduced susceptibility to chlorhexidine in staphylococci: is it increasing and does it matter?” J Antimicrob Chemother., 2012 November; 67(11): 2547-59), it is noted that different methods have been used for the detection of reduced susceptibility to chlorhexidine, but there is no standardized method and there is no consensus on the definition of ‘resistance’ to chlorhexidine. In particular in this publication, the evidence of reduced susceptibility to chlorhexidine in staphylococci was examined. The authors conclude that “the clinical use of chlorhexidine will continue to increase and it will be important to pay attention to the possibility that this could lead to the emergence of new clones with reduced susceptibility. The indiscriminate use of chlorhexidine in the absence of efficacy data should be discouraged”.

The object of the present invention is therefore to be able to benefit from the efficacy of chlorhexidine, while to avoid triggering mechanisms of resistance by the microorganisms concerned.

### SUMMARY OF THE INVENTION

The above object has been achieved by a composition comprising chlorhexidine or a salt thereof, and at least one peptide, as reported in claim 1.

The characteristics and the advantages of the present invention will become apparent from the following detailed description and from the working Examples provided for illustrative purposes.

### DETAILED DESCRIPTION OF THE INVENTION

The object of the invention therefore is a composition comprising chlorhexidine or a salt thereof, and at least one peptide, said at least one peptide consisting of 10-50 amino acids, wherein at least two amino acids are basic amino acids selected from Lys, His, Arg, or a combination thereof, and wherein at least 50% of the amino acids are hydrophobic amino acids.

In the composition of the invention, chlorhexidine is as a free base or in the form of a salt thereof; “salt of chlorhexidine” means dihydrochloride, diacetate, digluconate or a mixture thereof.

Preferably, the composition of the invention comprises up to 0.05 g/ml of chlorhexidine or a salt thereof and up to 12.5 ug/ml of said at least one peptide.

According to a preferred embodiment, the composition of the invention comprises up to 0.03 g/ml of chlorhexidine or a salt thereof and up to 6 ug/ml of said at least one peptide.

In some embodiments, said at least one cationic peptide is a peptide having a sequence A-B-C-D-C′-B′ -A′, where:
each unit A independently consists of 1-3 amino acids;
each unit B independently consists of a sulfur-containing amino acid;
each unit C independently consists of 5 amino acids selected from both the group (a) of hydrophobic amino acids and the group (b) of basic amino acids or hydrogen bond-forming amino acids;
unit D consists of glycine and a basic amino acid,
where:
   (i) said hydrophobic amino acids are selected from: Ala, Phe, Ile, Leu, Pro, Tyr, Trp and Val;
   (ii) said basic amino acids are selected from: Lys, His, Arg;
   (iii) said hydrogen bond-forming amino acids are selected from Asn, Gln, Ser, Thr; and where the substructure C-D-C′ contains a total of 5 to 9 points of alternation between amino acid of the group (a) and amino acid of the group (b), or vice versa.

It has in fact been surprisingly observed that the combination of chlorhexidine or a salt thereof with said at least one peptide generates an unexpected and very significant synergistic effect, as demonstrated in the Examples below. In particular, it has been shown that it is possible to use extremely low concentrations of chlorhexidine, as well as extremely low concentrations of peptide, to obtain more than satisfactory results, especially considering that at the same low concentrations the single components have shown no activity. The possibility of drastically reducing the concentration of chlorhexidine consequently drastically reduces the risk of triggering mechanisms of resistance by the microorganisms and the onset of side effects, with evident advantages not only from the economic and environmental points of view, but also and especially from the points of view of efficacy and safety of use.

Said at least one peptide has a preferable length of 15 to 21 amino acids, can be cyclized by formation of a disulfide bridge between two sulfur-containing amino acids, suitably located in the proximity of the —NH₂ terminal and -COOH terminal regions, in the cyclized form taking a twisted beta sheet shape. Furthermore, the central portion of the peptide is characterized by the presence of several charged amino acids, in part or totally alternating with neutral amino acids. More specifically, said peptide has a sequence of the A-B-C-D-C′-B′-A′ type, where: units A and A′ represent the —NH₂ terminal and —COOH terminal regions, respectively; units B and B′ consist of sulfur-containing amino acids; units C consist of 5 amino acids selected from: (a) hydrophobic amino acids and (b) basic or hydrogen bond-forming amino acids; unit D consists of a basic amino acid and glycine. Substructure C-D-C′ is characterized by containing a total of 5 to 9 points of alternation between amino acid of the group (a) and amino acid of the group (b), or vice versa.

The hairpin conformation of the peptide, due to the group D in the central portion of the sequence, juxtaposes the two sulfur-containing amino acids B, which in a suitable environment (air or oxidizing conditions) form the disulfide bridge. The cyclization of the structure contributes to the stability of the peptide and to the resistance to the action of bacterial peptidases, therefore, preferably said at least one peptide is in a cyclized form by formation of a disulfide bridge between the two units B.

The above-mentioned sequence comprises a percentage of hydrophobic amino acids so as not to perturb the membranes of the eukaryotic cells, while ensuring low/zero toxicity for such cells. Furthermore, the arrangement of the hydrophobic amino acids in discrete regions, separated by charged amino acids, i.e. basic and/or hydrogen bond-forming amino acids, imparts greater efficacy to the peptides and possibly also a higher salt-insensitivity. Finally, such peptides show a high solubility in aqueous solvents.

Said at least one peptide is easily synthesized, proteolytically stable, substantially salt-insensitive, non-hemolytic and non-cytotoxic to eukaryotic cells.

The term “peptide” is defined in the present invention as a plurality of amino acid residues linked by peptide bonds. It has the same meaning as polypeptide and protein and can be used interchangeably. The polypeptide-forming amino acids are identified herein without distinction either by their full name or by the relevant official international abbreviation (1 or 3 letter code).

The term “series” is defined as all possible variations of the at least one peptide wherein one or more amino acids of the peptide sequence are substituted with a homologous amino acid so that the properties of the peptides are maintained, though not necessarily at the same level. Another variant may have greater or lesser activity and/or a wider spectrum (for example, an activity against a wider range of microbes) or be more specific to a particular microorganism. Preferably, conservative substitutions of amino acids are carried out in one or more amino acid residues.

The term “beta sheet” refers to the three-dimensional structure of the cyclized peptide, where each strand has a clockwise twist of about 30′; such a geometry is the compromise between the conformational energy optimization of the two strands forming the sheet and the retention of the geometry of the intra-strand hydrogen bonds. In the above sequence, units A, B, C, D, C′, B′, A′ are connected in the order A-B-C-D-C′-B′-A′ to form a linear sequence; such a sequence can be cyclized or is cyclized by a direct bond between the two units B. In the above structure, the units marked with the same letter are not necessarily equal to each other but may contain different amino acids; it follows that, compared to the central group D, the invention includes both symmetric and asymmetric peptides.

Units A represent the peptide terminal regions: unit A indicates the —NH₂ terminal region while unit A′ indicates the —COOH terminal region. Each unit A and A′ independently consists of 1, 2 or 3 amino acids; preferably, moreover, the total number of amino acids in these two units A and A′ is equal to 3, or 2+1 or 1+2, respectively.

Preferably, unit A comprises at least one lysine and one amino acid selected from glycine, alanine, leucine, isoleucine, valine, tryptophan, histidine and arginine.

Preferably, unit A′ comprises at least one amino acid selected from glycine, alanine, leucine, isoleucine, valine, phenylalanine, tyrosine, tryptophan, histidine, arginine and lysine.

Units B denote a sulfur-containing amino acid, in particular cysteine or methionine. Units B are involved in the formation of the disulfide bridge responsible for the cyclization of the peptide. The cyclization may be carried out at the time of the synthesis of the peptide or it may occur later in the presence of an adequate environmental oxygen supply. Preferably, both units B and B′ are cysteine.

Unit D denotes a basic amino acid and glycine, preferably with: basic amino acid→glycine sequence, in the direction A→A′; the basic amino acid here is preferably arginine. The glycine present in D, usefully supported by arginine, allows the hairpin conformation of the peptide and, in combination with units C linked thereto, an adequate spacing of units B forming the disulfide bridge.

Units C consist, independently of each other, of 5 amino acids selected from:
(a) both the group of hydrophobic amino acids,
(b) and the group of basic or hydrogen bond-forming amino acids.

Hydrophobic amino acids of group (a) are selected from: alanine, phenylalanine, isoleucine, leucine, proline, tyrosine, tryptophan and valine. In relation to the total number of all amino acids of the peptide, they preferably represent between 30 and 50%, more preferably between 35 and 45%, for example between 39 and 43%.

The basic amino acids of group (b) are selected from: lysine, histidine, arginine.

The hydrogen bond-forming amino acids of group (b) are selected from asparagine, glutamine, serine, threonine.

Preferably, each unit A and A′ of the peptide independently consists of 1 or 2 amino acids and at least one of the units C and C′ comprises Lys.

More preferably, both units C and C′ of the peptide comprise Lys.

In a preferred embodiment, each unit C contains both amino acids of group (a) and amino acids of group (b); basic amino acids, hydrogen bond-forming amino acids or both may be freely used as members of group (b). In a preferred variant, units C contain 50-100% of basic amino acids as members of group (b).

An essential feature of units C is the high degree of alternation between the positively charged amino acids of group (b) and the electrically neutral amino acids of group (a). In particular, the sequence C-D-C′ contains 5 to 9 points of alternation between: (a) hydrophobic amino acid and (b) basic or hydrogen bond-forming amino acid, or vice versa. The number of “points of alternation” is equal to the number of peptide bonds which, in the sequence C-D-C′, separate an amino acid of group (a) from an amino acid of group (b) directly linked thereto: for example, the sequence Ala-His-Ala-Thr-Phe contains 4 points of alternation, corresponding to the 4 peptide bonds present in the sequence; conversely, a sequence Lys-Ala-Phe-Lys-Phe contains only 3 points of alternation: this is because Ala and Phe belong to the same class (a), and therefore the Ala-Phe bond does not count as “point of alternation”. For the purposes of the present invention, the “points of alternation” also include the bond between the basic amino acid present in D and the amino acid of unit C linked thereto, if said amino acid is a hydrophobic amino acid; conversely, the bonds involving glycine and the sulfur-containing amino acids do not count as “points of alternation”, irrespective of the amino acid linked thereto.

Therefore in units C, amino acids (a) and (b) are typically alternating; however, this does not exclude the possibility of limited adjacencies between amino acids of the same group ((a) or (b)), provided that said number of points of alternation in the sequence C-D-C′ is respected.

In a preferred embodiment, unit D of the peptide is -Arg-Gly-.

In a further preferred embodiment, the amino acid adjacent to Gly of unit D of the peptide is a hydrophobic amino acid, preferably an aromatic hydrophobic amino acid.

A preferred subgroup of peptides having the sequence described above is the subgroup containing a total of 17 amino acids, wherein the units A and A′ independently consist of 1 or 2 amino acids, the units B and B′ are both Cys, at least one of the units C and C′ comprises Lys, all the amino acids in position 6, 8, 13 (numbered from A to A′) belong to said group (i) of hydrophobic amino acids, and amino acids in position 9 and 10 are Arg and Gly.

A mostly preferred subgroup is characterized by containing, in addition to the characteristics listed above, a hydrophobic amino acid specifically in position 11 (always numbering from A to A′).

This characteristic is particularly useful to increase the salt-insensitivity of the peptide, i.e. the retention of its antibacterial action also in the presence of high concentrations of salt. This property is of particular importance since the membranolytic activity of antimicrobial peptides is generally based on the electrostatic interaction with the negatively charged bacterial or fungal membranes; normally, the presence of free ions (e.g. Na⁺, Cl⁻, commonly found in the assay medium or in the body/disease fluids) masks the negative charge present on the bacterial membrane, thus reducing the peptide binding efficiency and therefore the efficacy of treatment. The present peptides are not affected by this undesired phenomenon, thus keeping a significant efficacy (particularly against Gram-negative bacteria) in the presence of high environmental ion concentrations.

All the amino acids present in the at least one peptide may be present without distinction either in form D- or L-; preferably, they are mainly (i.e. more than 50%) or totally in the form L.

All the amino acids may be used in their natural state or in the form of synthetic derivatives thereof.

A preferred group of peptides is that in which:
unit A (—NH₂ terminal) contains 2 amino acids and unit A′ (—COOH terminal) contains 1 amino acid, and
both units B and B′ denote cysteine.

Specific preferred peptides according to the present invention are the peptide named AMP2041 (SEQ.ID. No.1), the peptide named AMP72 (SEQ.ID.No.2) and the peptide named AMP126 (SEQ.ID.No.3). Further peptides useful for the purposes of the invention are those of sequences SED.ID.No. 4-54 as described herein, in particular AMP289 (SEQ.ID.No.4), AMP944 (SEQ.ID.No.5), AMP573 (SEQ.ID.No.22), AMP1360 (SEQ.ID.No.8), AMP1189 (SEQ.ID.No.7), AMP1188 (SEQ.ID.No.6), AMP16 (SEQ.ID.No.9), AMP51 (SEQ.ID.No.10).

Preferred are also the nearby homologues of each of said SED.ID.No. 4-54, characterized by being modified in a single amino acid in any position between no. 1 and 17, where said modification does not affect the amino acids in position 3, 9, 10, 16; the change consists in replacing said amino acid with another amino acid selected from the 20 natural amino acids; preferably, the amino acid is substituted with another amino acid belonging to the same category (a) or (b) as defined above: for example, Ala is substituted with Leu; or Ser is substituted with Lys or Thr, etc.

Said at least one peptide is generally a synthetic peptide synthesized in vitro by using chemical methods known in the art. For example, it is prepared by using synthesis procedures on solid phase, liquid phase, peptide condensation or any combination of the above techniques. The amino acids, which form said at least one peptide, may be natural or synthetic. The amino acids used for the peptide synthesis may be amino acids wherein the α-amino-terminal is protected by the acid-labile group N-α-t-butyloxycarbonyl (Boc) according to Merrifield's work (J. Am. Chem. Soc., 85: 2149-2154,1963) or by the base-labile 9-fluorenylmethoxycarbonyl (Fmoc) as described by Carpino and Han (J. Org. Chem., 37:3403-3409, 1972). Both Boc- and Fmoc-protected amino acids can be obtained from commercial sources, such as Fluka, Sigma-Aldrich Bachem, Advanced Chemtech, Cambridge Biochemical Research.

In general, the methods of chemical synthesis on solid phase consist, according to M. Bodansky, ‘Principi di sintesi peptidica’ (Springer-Verlag, Berlin 1984) or J M Stewart and J D Young, ‘Solid Phase Peptide Synthesis’ (Pierce Chemical Co., Rockford, Ill. 1984), in the sequential addition of one or more amino acids to the growing peptide chain. Generally, the amino group or carboxyl group of the first amino acid is protected by an optimal protective group. The first protected amino acid is attached to a solid inert support such as a resin. The protective group is then removed from the resin-bonded residue and the subsequent amino acids (suitably protected) are added sequentially. After reaching the number of amino acids, all remaining protective groups (and any solid support) are removed sequentially or simultaneously, to have the final peptide.

More than one amino acid at a time may be added to the growing chain, for example by coupling (in suitable experimental conditions which prevent the formation of racemes, due to the presence of chiral centers) a protected tripeptide with a suitably protected dipeptide to form, after deprotection, a pentapeptide as described, for example, by Merrifield in G. Barany and R B Merrifield, ‘I peptidi: Analisi, Sintesi, Biologia’, Ed. E. and J. Gross Meienhofer, vol. 2, (Academic Press, New York, 1980, pp 3-254).

Said peptides can be synthesized by companies providing the custom peptide synthesis service, such as, but not limited to, Sigma-Aldrich (St. Louis, Mo., USA), SelleckChem (Houston, Tex., USA), Invitrogen (Grand Island, N.Y., USA), Abgent, OX144RY, Oxfordshire (United Kingdom). The degree of purity of the peptide compound can be determined by various methods, including the identification of HPLC peaks. Preferably, a peptide which produces a single peak of height and width of at least 75% of the incoming material on an HPLC column is preferred. Even more preferred is a peptide which produces a single peak which is at least 87%, at least 90%, at least 99% or even 99.5% of the incoming material on an HPLC column.

To ensure that the peptide obtained by using one of the above synthesis techniques is the desired peptide for the uses or the formulations described hereafter in the present invention, the analysis of the composition of the peptide is carried out with the aid of different analytical methods known in the art. The analysis of the composition can be carried out, for example, by using the high resolution mass spectrometry to determine the molecular weight of the peptide. Alternatively, the amino acid contents in a peptide can be confirmed by hydrolyzing the peptide in acidic solution to identify and quantify the components of the mixture using HPLC, or an amino acid analyzer. Equally useful are thin layer chromatographic methods, which may also be used to identify one or more constituent groups or residues of a desired peptide.

Another preferred aspect of the at least one peptide relates to the polar angle between 90° and 180°, preferably between 91° and 179°, more preferably between 104° and 115°. The term “polar angle” means, in the present document, the measure of the angle formed between the polar and non-polar side of a peptide conformed in an amphiphilic structure.

Another preferred aspect of the at least one peptide relates to the Boman index between −1 and +4. Preferably, between −0.5 and +3 and even more preferably between +1 and +2.5, for example between +1.1 and +2.0. The term “Boman index” is defined in the present invention as the sum of the transfer energies from water to the cyclohexane of the side chains of the single amino acids forming the peptides divided by the total number of residues, according to what described by

Radzeka and Wolfenden (1988) in “Comparing the polarities of amino acids: side-chain distribution coefficients between vapor phase, cyclohexane, 1-octanol and neutral aqueous solution.” (Biochemistry 27:1664-1670). The calculated values are negative but the sign (+or −) is reversed.

Another preferred aspect relates to the percentage of solubility in water of the at least one peptide in the range between 40% and 90%, preferably between 91% and 97%, even more preferably between 97.5% and 100%, such as 98%. The estimated percentage of solubility is calculated by using the two-parameter solubility model of Wilkinson-Harris, as described in Wilkinson D L and Harrison R G (1991) Bio/Technology 9, 443-448.

In other embodiments, said at least one peptide is human Beta-defensin 1 (hBD1), human Beta-defensin 2 (hBD2), human Beta-defensin 3 (hBD3), human Beta-defensin 4 (hBD4), LL-37, Lactoferricin B, Lactoferrin (f 17-41), Temporin A, Temporin B, Temporin L, Indolicin, Melittin, Protegrin-1, Protegrin-2, Protegrin-3, Protegrin-4, Protegrin-5, Magainin 2, RTD-1, RTD-2, RTD-3, RTD-4, RTD-5, Arenicin-1, Arenicin-2 Arenicin-3, Dermcidin, Cecropin, Andropin, Moricin, Ceratotoxin, Dermaseptin, Bombinin, preferably Maximin H1, Maximin H2, Maximin H3, Maximin H4 or Maximin H5, Esculentin, Ranalexin, Buforin II, human CAP18, Abaecin, Apidaecin, Profenin, Bactenecin, Brevinin-1, Brevinin-2, Tachyplesin, or Drosomycin. ‘RTD’ stands for Rhesus Theta-Defensin.

Preferably, said at least one peptide is human Beta-defensin 1 (hBD1), human Beta-defensin 2 (hBD2), human Beta-defensin 4 (hBD4), LL-37, Lactoferricin B, Lactoferrin (f 17-41), Temporin A, Temporin B, Temporin L, Indolicin, Melittin, Protegrin-1, Protegrin-2, Protegrin-3, Protegrin-4, Protegrin-5, Magainin 2, RTD-1, RTD-2, RTD-3, RTD-4, RTD-5, Arenicin-1, Arenicin-2 Arenicin-3, Dermcidin, Cecropin, Andropin, Moricin, Ceratotoxin, Dermaseptin, Bombinin, preferably Maximin H1, Maximin H2, Maximin H3, Maximin H4 or Maximin H5, Esculentin, Ranalexin, Buforin II, human CAP18, Abaecin, Apidaecin, Profenin, Bactenecin, Brevinin-1, Brevinin-2, Tachyplesin, or Drosomycin.

More preferably, said at least one peptide is human Beta-defensin 3 (hBD3), LL-37, Lactoferricin B, Lactoferrin (f 17-41), Temporin A, Indolicin, Melittin, Protegrin-1, Magainin 2, Arenicin-1, Arenicin-2, Arenicin-3 or RTD-5.

Preferably, in these other embodiments, chlorhexidine or a salt thereof is in a concentration from 0.8 ug/ml to 0.05 g/ml.

In further embodiments, the composition of the invention comprises chlorhexidine or a salt thereof, at least one peptide selected from human Beta-defensin, LL-37, Lactoferricin B, Lactoferrin (f 17-41), Temporin A, Temporin B, Temporin L, Indolicin, Melittin, Protegrin-1, Protegrin-2, Protegrin-3, Protegrin-4, Protegrin-5, Magainin 2, RTD-1, RTD-2, RTD-3, RTD-4, RTD-5, Arenicin-1, Arenicin-2 Arenicin-3, Dermcidin, Cecropin, Andropin, Moricin, Ceratotoxin, Dermaseptin, Bombinin, preferably Maximin H1, Maximin H2, Maximin H3, Maximin H4 or Maximin H5, Esculentin, Ranalexin, Buforin II, human CAP18, Abaecin, Apidaecin, Profenin, Bactenecin, Brevinin-1, Brevinin-2, Tachyplesin, or Drosomycin, and at least one cationic peptide having a sequence A-B-C-D-C′-B′-A′, as described above.

In other preferred embodiments, the composition of the invention further comprises a buffer solution comprising a buffer compound selected from TRIS (or tris(hydroxymethyl)aminomethane), PIPES (or piperazin-1,4-bis (2-ethanesulfonate acid)), TRIS*HCl, HEPES (or 4-2-hydroxyethyl-1-piperazinyl-ethanesulfonic acid), sodium phosphate monobasic and dibasic acid, or citric acid, and comprising a sequestering agent selected from EGTA (ethyleneglycoltetraacetic acid), EDTA (ethylenediaminetetraacetic acid) or an anhydrous or hydrated-salt form thereof, calcium disodium EDTA or a hydrated form thereof, diammonium EDTA or a hydrated form thereof, dipotassium EDTA or a hydrated form thereof, disodium EDTA or a hydrated or dihydrated form thereof, TEA-EDTA (EDTA salt of mono (triethanolamine)), tetrasodium EDTA, tripotassium EDTA, trisodium EDTA, HEDTA (hydroxyethyl-ethylenediaminotriacetic acid), HEDTA-EDTA, and mixtures thereof.

Suitable concentrations of buffer compound are up to 1 g/ml and suitable concentrations of sequestering agent are up to 0.5 g/ml.

Preferably, when the at least one peptide is a cationic peptide having a sequence A-B-C-D-C′-B′-A′, the composition of the invention comprises up to 0.0025 g/ml of chlorhexidine or a salt thereof, up to 10.0 ug/ml of at least one peptide, up to 0.5 g/ml of buffer compound and up to 0.2 g/ml of sequestering agent. More preferably, the composition of the invention comprises up to 0.002 g/ml of chlorhexidine or a salt thereof, up to 5.0 ug/ml of at least one peptide selected from SEQ.ID.No. 1-54, up to 0.1 g/ml of buffer compound, and up to 0.01 g/ml of sequestering agent.

When said at least one peptide is selected from one of the other peptides described above, preferably the composition of the invention comprises from 0.8 ug/ml to 0.0025 g/ml of chlorhexidine or a salt thereof, up to 10.0 ug/ml of at least one peptide, up to 0.5 g/ml of buffer compound and up to 0.2 g/ml of sequestering agent.

In a particularly preferred embodiment, said buffer solution comprises TRIS and EDTA disodium dihydrate.

As will be seen in the following examples, the further presence of the buffer solution surprisingly allows a further increase in the efficacy and activity of the composition itself.

In another aspect thereof, the invention relates to the use of the composition of the invention for the manufacture of a medicament for treating a subject suffering from an infection caused by bacteria, fungi and/or yeasts; the invention also extends to the same composition for use in the treatment of an infection caused by bacteria, fungi and/or yeasts. In a first variant, the treatment is directed in particular against the group of gram-negative bacteria; in a second variant, the treatment is directed against the group of gram-positive bacteria; in a third variant, the treatment is directed against the group of fungi and yeasts; in a fourth variant, the treatment is directed against microorganisms belonging to one or more of the above groups. The term “treatment” refers to the effects of the composition of the invention, which is able to impart a benefit to patients suffering from an infectious disease, such as an improvement in the patient's condition or delay in the progression of the disease. In the present document, the term “infection” or its synonym “infectious disease” refers to the invasion, colonization and/or multiplication of a microorganism into or on another host organism. The term “microbial infection” means an infectious disease caused by a pathogen, defined above, e.g. a bacterium, a parasite, a protozoan, a virus or a fungus, including yeasts. In the present document, the term “subject” defines any multicellular organism, including a human being, an animal, an insect or a plant, which may be infected with a microorganism. Preferably, the subject is any animal organism, such as a human being or animal, which may be infected with a microorganism against which an antimicrobial peptide or a variant thereof is active.

A pathogenic bacterium, as defined above, can result from one of the bacterial species such as: Staphylococcus spp, e.g. Staphylococcus aureus (e.g. Staphylococcus aureus ATCC 25923), Enterococcus spp, e.g. Enterococcus faecalis ATCC 29212; Pseudomonas spp., e.g. Pseudomonas aeruginosa ATCC 27853; Mycobacterium spp, e.g. Mycobacterium tuberculosis; Enterobacter spp; Campylobacter spp; Salmonella spp (e.g. Salmonella enteritidis ATCC13076); Streptococcus spp, e.g. Streptococcus group A or B, Streptoccocus pneumoniae, Helicobacter spp, e.g. Helicobacter pylori; Neisseria spp, e.g. Neisseria gonorrea, Neisseria meningitidis; Borrelia burgdorferi, Shigella spp, e.g. Shigella flexneri; Escherichia coli (ATCC 25922); Haemophilus spp, e.g. Haemophilus influenzae; Francisella tularensis, Bacillus spp, e.g. Bacillus anthracis; Clostridium spp, Clostridium botulinum, Yersinia spp, e.g. Yersinia pestis; Treponema spp; Burkholderia spp; e.g. Burkholderia cepacia ATCC 17759, B. mallei and B. pseudomallei; Stenotrophomonas spp, e.g. Stenotrophomonas maltophilia ATCC 13637.

The biological activity of the composition of the invention against microorganisms was determined, for example, on gram-negative bacteria, with reference to bacteria such as Pseudomonas aeruginosa. In particular, Pseudomonas aeruginosa is a problematic gram-negative bacterium due to its invasiveness and heterogeneous resistance to antibacterial chemotherapy. This microorganism is responsible for severe infections and causes significant morbidity in subjects immunocompromised from viral infections such as HIV, cancer chemotherapy or immunosuppressive therapies. Moreover, this bacterium is often the causative agent of severe infectious diseases of the lower respiratory tract, urinary tract, skin lesions (sores, ulcers) in young people, including subjects suffering from cystic fibrosis, and elderly hospitalized patients. In recent years, the incidence of Pseudomonas infections in cystic fibrosis has been significantly increasing. A fungal pathogen can be derived from one of the fungi (including yeasts) belonging to the group comprising the geni Candida spp. (e.g. C. albicans), Epidermophyton spp. Exophiala spp. Microsporum spp. Trichophyton spp. (e.g. T. rubrum e T. interdigitale), Tinea spp. Aspergillus spp. Blastomyces spp. Blastoschizomyces spp. Coccidioides spp. Cryptococcus spp. (e.g. Cryptococcus neoformans), Histoplasma spp. Paracoccidiomyces spp. Sporotrix spp. Absidia spp. Cladophialophora spp. Fonsecaea spp. Phialophora spp. Lacazia spp. Arthrographis spp. Acremonium spp. Actinomadura spp. Apophysomyces spp., Emmonsia spp. Basidiobolus spp. Beauveria spp. Chrysosporium spp. Conidiobolus spp. Cunninghamella spp. Fusarium spp. Geotrichum spp. Graphium spp. Leptosphaeria spp. Malassezia spp. (e.g. Malassezia furfur), Mucor spp. Neotestudina spp. Nocardia spp., Nocardiopsis spp. Paecilomyces spp. Phoma spp. Piedraia spp. Pneumocystis spp. Pseudallescheria spp. Pyrenochaeta spp. Rhizomucor spp. Rhizopus spp. Rhodotorula spp. Saccharomyces spp. Scedosporium spp. Scopulariopsis spp. Sporobolomyces spp. Syncephalastrum spp. Trichoderma spp. Trichosporon spp. Ulocladium spp. Ustilago spp. Verticillium spp. Wangiella spp.

In a preferred variant, the treatment is directed against a microorganism selected from Pseudomonas spp., Escherichia spp, Stenotrophomonas spp., Burkholderia spp., Candida spp. or Malassezia spp.

The composition of the present invention may also be useful in the treatment of infections usually associated with the skin, such as ulcers and lesions and skin wounds, cuts or burns.

A further preferred aspect of the invention indicates that the composition is useful in the treatment of bacterial skin infections or pyodermite.

Another aspect involves the use of the composition of the invention in the treatment of (clinical or surgical) diseases complicated by bacterial suprainfections, such as infections associated with the mucosa, infections associated with the gastrointestinal, genitourinary tract, infections of the urinary tract (e.g. pyelonephritis, cystitis, urethritis) or respiratory infections, for example cystic fibrosis. Mammals, birds and, in general, other animals can be treated with the peptides described in the present invention. Mammals and birds include, but are not limited to, humans, dogs, cats and pet birds and productive livestock such as horses, cattle, sheep, goats, pigs, chickens and turkeys and poultry.

A second preferred aspect involves the use of the composition of the invention in the treatment of infectious diseases: infections from Klebsiella, Salmonella, Yersinia, Proteus and Colibacillosis of pets and productive livestock.

Another preferred aspect relates to the treatment of glanders in equidae and melioidosis in carnivores and Pseudomonas aeruginosa infections in pets and productive livestock.

Another aspect relates to the treatment of Bordetella spp infections in pet animals and productive livestock; Moraxella spp infections; Francisella spp infections, Brucella spp infections, Campylobacter spp. infections, Pasteurella spp. infections; Actinobacillus spp. infections (Actinobacillosis); Haemophilus spp. infections; Streptococcus spp. infections (including mastitis in cattle, strangles); Staphylococcus spp. infections (including mastitis, pyoderma, endometritis); Bacillus spp. infections, including anthrax; Clostridium spp infections, including tetanus, botulism and symptomatic anthrax; Listeria spp. infections (listeriosis); Erysipelothrix spp. infections, including erysipelas suis; Leptospira spp. infections, Serpulina (surface necrotic enteritis), Treponema spp. (rabbit syphilis), Borrelia spp. in pet animals and productive livestock.

Finally, also plants can be treated with the composition of the invention.

The composition of the invention may be prepared by procedures described in the art and with excipients known and readily available. Such compositions form part of the present invention.

In the present document, the term “excipient” means a compound or an optimal mixture thereof for use in a formulation prepared for the treatment of a specific infectious disease or conditions associated therewith. For example, an excipient for use in a pharmaceutical formulation must not generally cause an adverse response in a subject. The excipient, as described above, should not significantly inhibit the relevant biological activity of the active compound. For example, an excipient does not significantly inhibit the antimicrobial activity of the composition of the present invention or a variant thereof.

Suitable excipients are sweeteners, diluents, disintegrants, glidants, coloring agents, binders, lubricants, stabilizers, adsorbents, preservatives, surfactants, humectants, perfumes, sebo-reducing agents, keratinolitic agents, softeners, restructuring agents, film-forming substances, emulsifiers, wetting agents, release retardants or mixtures thereof.

Preferably, the surfactants are amphoteric surfactants in a concentration not higher than 15% (0.15 g/ml), non-ionic surfactants in a concentration not higher than 15% (0.15 g/ml) or cationic surfactants in a concentration not higher than 15% (0.15 g/ml).

Emulsifiers, humectants, sebo-reducing agents, keratinolitics, softeners, restructuring agents are preferably in a concentration not higher than 15% (0.15 g/ml).

Lubricants and film-forming agents are preferably in a concentration not higher than 3% (0.03 g/ml).

Preservatives are preferably in a concentration not higher than 1% (0.01 g/ml).

Coloring agents are preferably in a concentration not higher than 2% (0.02 g/ml).

Perfumes are preferably in a concentration not higher than 5% (0.05 g/ml).

Alternatively or in addition, the excipient can comprise a compound, such as a protease inhibitor, which increases the activity or half-life of the at least one peptide. In another example, the excipient may include or be itself an additional antimicrobial molecule and/or an anti-inflammatory molecule.

The composition of the invention may also take the form of an aqueous solution, an anhydrous form or a dispersion, or alternatively the form of an emulsion, a suspension, an ointment, a cream, a paste, a gel or a salve.

The composition of the invention may also take the form of a topical product for cleansing and cleaning of both human beings and animals, such as a gel, a spray, an aqueous solution, a shampoo or an otological or dermatological solution.

Said at least one peptide of the composition of the present invention may be formulated in powder form, obtained by aseptic isolation of a sterile solid or by lyophilization of a solution to be reconstituted in the form of solution with the aid of a suitable carrier prior to use, for example water.

The composition may be in solid form, such as tablet, mini-tablet, micro-tablet, granule, micro-granule, pellet, multiparticulate, micronized particulate, or it may be in the form of a solution, cream, ointment, salve, paste, oil, emulsion, gel, vials or drops.

The solid forms, such as tablets, may be coated with standard aqueous or non-aqueous techniques.

The amount of active compounds in such therapeutically useful compositions is such as to allow a therapeutically effective dosage to be obtained. The active compounds may also be administered by auricular route, for example liquid drops or spray.

The solid forms may also contain a binder such as tragacanth gum, acacia, corn starch or gelatin; excipients such as dicalcium phosphate; a disintegrating agent such as corn starch, potato starch, alginic acid; and a lubricant such as magnesium stearate.

Controlled-release, slow-release or sustained-release forms may be provided.

A liquid form may contain, in addition to the active compounds, methyl and propyl paraben as preservatives, a dye and a flavoring agent.

Compositions for topical administration include ointments, creams, lotions, solutions, pastes, gels, liposomes, nanoparticles, patches, bandages and dressings. In certain embodiments, the topical formulation comprises at least one cutaneous penetration promoter.

The administration of the compositions is carried out at a dosage sufficient to produce the desired therapeutic effect in the subject.

In some embodiments, the effective amounts for topical formulation will depend on the severity of the disease, disorder or condition, previous therapy, the state of health of the individual and the response to the composition.

The composition of the invention may further advantageously be used as an antibacterial, antifungal or anti-yeast agent, for the disinfection and sanitization of surfaces or supports.

In fact, it was surprisingly found that the composition of the invention allows significantly satisfactory results to be obtained in the disinfection and sanitization of surfaces and supports, in particular of equipment and environments for veterinary use, at the same time without damaging the same, so as to suitably prevent the development of infections and diseases from contact also in animals which use such equipment and environments.

Preferably, they are equipment and environments of the dairy field, such as milking buckets, bins, filters, milking machines, tanks, pipes, filling machines, glassware, cream separators, tanks, boilers, cold storage tanks and reservoirs, milk processing lines, surfaces and floors. Said surfaces and supports may be made of steel, metals, polymers, such as polyethylene and PVC, elastomers or combinations thereof.

To this end, the composition of the invention can advantageously be in solid form, as described above, so as to achieve extremely and significantly increased storage times compared to corresponding liquid forms, as well as a convenient reduction in the storage volume.

Preferably, the composition of the invention for the disinfection and sanitization of surfaces or supports comprises at least one peptide selected from LL-37, Temporin A, Temporin B, Temporin L, Melittin and Magainin 2.

It is understood that any possible combinations of the preferred aspects of the components of the composition as indicated above are likewise described and therefore preferred.

It is also to be understood that all aspects identified as preferred and advantageous for the composition and its components are to be deemed as similarly preferred and advantageous also for the preparation and use of the same.

Below are working examples of the present invention provided for illustrative purposes.

### EXAMPLES

### Example 1

### Peptides

The peptides used in the experimental plan are AMP2041, AMP126 and AMP72. They are synthesized by SelleckChem (Houston, Tex., USA) and are characterized by a purity of not less than 90%. The freeze-dried peptides are dissolved in phosphate buffer (PB, 10 mM, 0.8709 g/L K₂HPO₄, 0.6804 g/L KH₂PO₄) at a concentration of 1 mg/ml.

### Preparation of the Bacterial Suspension

For each strain of reference, three to five morphologically similar colonies are selected from blood agar plates and inoculated into tubes containing 6 ml of Brain Heart Infusion broth (DIFCO, USA).

The tubes are vortexed and incubated at 37° C. with stirring at 225 rpm for 3-4 hours, until achieving a required degree of turbidity, greater than or equal to 0.5 of the Mc Farland scale.

Thereafter, the bacterial suspension is centrifuged at 1000 rpm for 20 minutes. The resulting pellet of bacteria is resuspended in 10mM PB. The turbidity is adjusted with the same buffer solution and checked by measuring the absorbance of the suspension at the spectrophotometer. The absorbance is considered equal to 0.5 of the Mc Farland scale when the optical density at 600 nm is between 0:08-0:13. At this value, the bacterial suspension contains approximately 10⁸UFC/ml. Two hundred microliters of the assessed and adjusted bacterial suspension are added to 19.8 ml of 10 mM PB to obtain a final dilution of 1:100. Then, within a thirty minutes, 0:05 ml of this suspension (10⁶UFC/ml) are inoculated into each well of an ELISA plate to obtain a final concentration of bacteria of about 5×10⁵UFC/ml.

(CSLI. Performance standards for antimicrobial disks and dilution susceptibility test form isolated from animals.; CLSI: Wayne, Pa., USA, 2008.)

### Preparation of the Plates and Evaluation of the Minimum Bactericidal Concentration (MBC).

Fifty microliters of the peptide 400 μg/ml are added to each well of column 1 of a microtiter plate containing 50 microliters of the buffer solution (PB). Serial dilutions are then carried out from column 1 to column 10 to obtain concentrations of peptide between 200 μg/ml and 0.4 μ,g/ml. Growth and sterility controls are inserted in column 11 and 12, respectively. 50 μl of work bacterial suspension are added to each well, with the exception of the sterility controls, to obtain a final concentration of the peptide of between 100 μg/ml and 0.2 μg/ml. The plate is incubated for 2 hours at 37° C. in air. Subsequently, 20 μl of each dilution are plated on a suitable solid medium and incubated for 24 h at 37° C. to proceed to the count of CFU. The Minimum Bactericidal Concentration (MBC) is considered as the lowest concentration able to kill at least 99.9% of bacteria. All experiments were repeated in triplicate.

### Time-Course of the Bactericidal Activity of the Peptides

1×10⁶ CFU of bacterial suspension in exponential growth are resuspended in 100 μl of 10 mM PB and brought into contact with the peptides AMP72, AMP126 and AMP 2041 to values close to the MBC and incubated at 37° C. Subsequently, 20 μl are seeded at different time intervals (every 5′ to 30′, then every 10′ to 60′, then, every 30′ to 120′) on specific media for the different bacterial species being examined. The growth controls are set up in the PB in the absence of peptide and seeded on solid medium at each time interval for counting the CFU. Finally, after an overnight incubation at 37° C., the colonies are counted. All experiments were repeated in triplicate.

### Evaluation of the Bactericidal Activity of New Formulations: Time-Killing Assay

The time-killing assay was carried out to evaluate the bactericidal effects of the new formulations, using a modified quantitative test of the European standards EN1040 and EN1276. 100 μl of the bacterial suspension are added to 900 μl of the new formulation and aliquots of 50 μl of the mixture are subjected to assay at 0.5, 1, 2.5, 5, 10, 20, 30, 60, 90 and 120 min., respectively. 450 μl of neutralizing solution are added to each sample and left to act at room temperature for 3 minutes. Thereafter, each sample is serially diluted in PBS (pH 7.4) and 50 μl are plated on LB agar and the plates incubated at 37° C. for 24 hours for the count of CFU. The sterility control (100 μl PBS in place of the bacterial suspension) and the control in the absence of the new formulation are set up. The test is also carried out in the presence of 0.3% and 3.0% (w/v) BSA to simulate a paraphysiological environment. All experiments were repeated in triplicate.

### RESULTS

The following table gives the results of the tests carried out. In particular, the activity of the single components, namely chlorhexidine (CLX) and peptide AMP 2041, their combination according to the present invention as well as a preferred combination also comprising TRIS-EDTA, was compared.

These tests clearly show the synergistic effect of the chlorhexidine+peptide combination in terms of minimum concentrations observed against the Pseudomonas aeruginosa ATCC 27853 bacterium.

| Pseudomonas aeruginosa | CC | T0 | T0.5 | T1 | T3 | T5 | T10 |
|---|---|---|---|---|---|---|---|
| CLX 0.000106% | 600 | 600 | 600 | 600 | 600 | 600 | 600 |
| | | Δ = 0% | Δ = 0% | Δ = 0% | Δ = 0% | Δ = 0% | Δ = 0% |
| AMP 2041 0.5 μg/ml | 600 | 600 | 600 | 600 | 600 | 600 | 600 |
| | | Δ = 0% | Δ = 0% | Δ = 0% | Δ = 0% | Δ = 0% | Δ = 0% |
| TRIS-EDTA 0.1% | 600 | 600 | 600 | 600 | 600 | 600 | 600 |
| | | Δ = 0% | Δ = 0% | Δ = 0% | Δ = 0% | Δ = 0% | Δ = 0% |
| CLX 0.000106% + | 600 | 600 | 480 | 300 | 180 | 108 | 42 |
| AMP 2041 0.5 μg/ml | | Δ = 0% | Δ = −20% | Δ = −50% | Δ = −70% | Δ = −82% | Δ = −93% |
| CLX 0.000106% + | 600 | 600 | 300 | 150 | 46 | 14 | 0 |
| AMP 2041 0.5 μg/ml + | | Δ = 0% | Δ = −50% | Δ = −75% | Δ = −92.3% | Δ = −97.7% | Δ = −100% |
| TRIS-EDTA (0.476%- | | | | | | | |
| 0.126%) | | | | | | | |

where T0=0 min, T0.5=0.5 min, T1=1 min, T2.5=2.5 min, T5=5 min, T10=10 min

As clear from the above Table, the surprising synergistic effect on Pseudomonas aeruginosa ATCC 27853 observed for the ‘chlorhexidine+peptide’ combination is advantageously further increased by the presence of TRIS-EDTA.

The same protocol was also applied to test the antibacterial activity against the Staphylococcus ATCC 25923 bacterium. The following table shows the results of the tests carried out. Likewise, the activity of the single components, namely chlorhexidine (CLX) and peptide AMP 2041, and of their combination according to the present invention, was compared.

| Staphylococcus aureus | CC | T0 | T0.5 | T1 | T3 | T5 | T10 |
|---|---|---|---|---|---|---|---|
| CLX 0.000106% | 600 | 600 | 600 | 600 | 600 | 600 | 600 |
| | | Δ = 0% | Δ = 0% | Δ = 0% | Δ = 0% | Δ = 0% | Δ = 0% |
| AMP 2041 0.5 μg/ml | 600 | 600 | 600 | 600 | 600 | 600 | 600 |
| | | Δ = 0% | Δ = 0% | Δ = 0% | Δ = 0% | Δ = 0% | Δ = 0% |
| CLX 0.000106% + | 600 | 600 | 600 | 250 | 100 | 4 | 0 |
| AMP 2041 0.5 μg/ml | | Δ = 0% | Δ = 0% | Δ = −58% | Δ = −83% | Δ = −99% | Δ = −100% |

where T0=0 min, T0.5=0.5 min, T1=1 min, T2.5=2.5 min, T5=5 min, T10=10 min

It was therefore surprisingly observed that the combination of the invention, ever since 1 minute after, considerably (50-60%) reduced the CFU of both gram-negative bacteria and of gram-positive bacteria. In particular, it was observed that, ever since only 5 minutes after, the reduction was almost complete (99.33%).

### Examples 2-17

Below is the preparation of products made according to the present invention. “%” concentration, in the following examples, means “g/100 ml”.

### 2A-2L. Preparation of Otological Veterinary Products

Products (with 8 pH or pH 7-7.5) for cleaning and disinfection of the ear were prepared, having the following composition:

| Ingredients | % concentration |
|---|---|
| demineralized water | 89.47995 |
| dihydrated disodium EDTA | 0.1 |
| tris-hydroxymethyl-aminomethane | 0.4 |
| propylene glycol | 10 |
| chlorhexidine digluconate | 0.02 |
| peptide | 0.00005 |

wherein “peptide” is:
Example 2A: AMP2041
Example 2B: AMP72
Example 2C: AMP126
Example 2D: human Beta-defensin 3 (hBD3)
Example 2E: Lactoferrin (f 17-41)
Example 2F: Arenicin-1
Example 2G: RTD-5
Example 2H: AMP2041+human Beta-defensin 3 (hBD3) (1:1)
Example 2I: AMP72+Lactoferrin (f 17-41) (1:1)
Example 2L: AMP289

### 3A-3L. Preparation of Liquid Veterinary Shampoos

Liquid shampoos (with pH 5.5-6.5) were prepared, having the following composition:

| Ingredients | % concentration |
|---|---|
| demineralized water | 69.45 |
| dihydrated disodium EDTA | 0.4 |
| tris-hydroxymethyl-aminomethane | 1.6 |
| isodecyl alcohol | 9 |
| aminoxide | 11.25 |
| ethoxylated lanolin | 4 |
| isopropyl alcohol | 2.5 |
| perfumed essence | 0.4 |
| color | 0.001 |
| chlorhexidine digluconate | 0.08 |
| peptide | 0.0002 |

wherein “peptide” is:
Example 3A: AMP2041
Example 3B: AMP72
Example 3C: AMP126
Example 3D: Lactoferricin B
Example 3E: Protegrin-1
Example 3F: Indolicin
Example 3G: RTD-5
Example 3H: AMP2041+Indolicin (2:1)
Example 3I: AMP126+Protegrin-1 (1:2)
Example 3L: AMP944

### 4A-4L. Preparation of Liquid Veterinary Shampoos

Liquid shampoos (with pH 5.5-6.5) were prepared, having the following composition:

| Ingredients | % concentration |
|---|---|
| demineralized water | 84.65 |
| dihydrated disodium EDTA | 0.4 |
| tris-hydroxymethyl-aminomethane | 1.6 |
| isodecyl alcohol | 0.55 |
| aminoxide | 8 |
| ethoxylated lanolin | 4 |
| isopropyl alcohol | 0.4 |
| perfumed essence | 0.4 |
| color | 0.001 |
| chlorhexidine digluconate | 0.08 |
| peptide | 0.0002 |

wherein “peptide” is:
Example 4A: AMP2041
Example 4B: AMP72
Example 4C: AMP126
Example 4D: Lactoferricin B
Example 4E: Protegrin-1
Example 4F: Indolicin
Example 4G: RTD-5
Example 4H: AMP2041+Indolicin (2:1)
Example 4I: AMP126+Protegrin-1 (1:2)
Example 4L: AMP944

### 5A-5L. Preparation of Liquid Veterinary Shampoos

Liquid shampoos (with pH 5.5-6.5) were prepared, having the following composition:

| Ingredients | % concentration |
|---|---|
| demineralized water | 69.471 |
| dihydrated disodium EDTA | 0.4 |
| tris-hydroxymethyl-aminomethane | 1.6 |
| 30% betaine sol. | 3.33 |
| aminoxide | 20 |
| ethoxylated lanolin | 4 |
| isopropyl alcohol | 0.799 |
| perfumed essence | 0.4 |
| color | 0.001 |
| chlorhexidine digluconate Staphylococcus | 0.08 |
| aureus ATCC 27300 | |
| peptide | 0.0002 |

wherein “peptide” is:
Example 5A: AMP2041
Example 5B: AMP72
Example 5C: AMP126
Example 5D: Lactoferricin B
Example 5E: Protegrin-1
Example 5F: Indolicin
Example 5G: RTD-5
Example 5H: AMP2041+Indolicin (2:1)
Example 5I: AMP126+Protegrin-1 (1:2)
Example 5L: AMP944

### 6A-6L. Preparation of Viscous Veterinary Shampoos

Liquid shampoos (with pH 7-9) were prepared, having the following composition:

| Ingredients | % concentration |
|---|---|
| demineralized water | 68.471 |
| cellulose | 1 |
| dihydrated disodium EDTA | 0.4 |
| tris-hydroxymethyl-aminomethane | 1.6 |
| 30% betaine sol. | 3.33 |
| aminoxide | 20 |
| ethoxylated lanolin | 4 |
| isopropyl alcohol | 0.799 |
| perfumed essence | 0.4 |
| color | 0.001 |
| chlorhexidine digluconate | 0.08 |
| peptide | 0.0002 |

wherein “peptide” is:
Example 6A: AMP2041
Example 6B: AMP72
Example 6C: AMP126
Example 6D: Lactoferricin B
Example 6E: Protegrin-1
Example 6F: Indolicin
Example 6G: RTD-5
Example 6H: AMP2041+Indolicin (2:1)
Example 6I: AMP126+Protegrin-1 (1:2)
Example 6L: AMP944

### 7A-7L. Preparation of Disinfectant Solutions

Disinfectant solutions (with pH 5.5-6.5) were prepared, having the following composition:

| Ingredients | % concentration |
|---|---|
| water | 86.4 |
| dihydrated disodium EDTA | 0.1 |
| tris-hydroxymethyl-aminomethane | 0.4 |
| isotridecanol ethoxylate | 0.45 |
| glycerin | 7.5 |
| propylene glycol | 5 |
| chlorhexidine digluconate | 0.02 |
| peptide | 0.00005 |

wherein “peptide” is:
Example 7A: AMP2041
Example 7B: AMP72
Example 7C: AMP126
Example 7D: Lactoferrin (f 17-41)
Example 7E: Arenicin-2
Example 7F: Esculentin
Example 7G: AMP573
Example 7H: AMP126+Lactoferrin (f 17-41) (1:1)
Example 7I: AMP72+Arenicin-2 (1:1)
Example 7L: AMP1360

### 8A-8L. Preparation of Disinfectant Solutions

Disinfectant solutions (with pH 5.5-6.5) were prepared, having the following composition:

| Ingredients | % concentration |
|---|---|
| water | 98.8215 |
| dihydrated disodium EDTA | 0.1 |
| tris-hydroxymethyl-aminomethane | 0.4 |
| isodecyl alcohol | 0.225 |
| aminoxide | 0.281 |
| ethoxylated lanolin | 0.1 |
| isopropyl alcohol | 0.0625 |
| chlorhexidine digluconate | 0.02 |
| glycerin | 2 |
| propylene glycol | 2 |
| peptide | 0.00005 |

wherein “peptide” is:
Example 8A: AMP2041
Example 8B: AMP72
Example 8C: AMP126
Example 8D: AMP16
Example 8E: Protegrin-2
Example 8F: human Beta-defensin 3 (hBD3)
Example 8G: AMP1189
Example 8H: AMP2041+human Beta-defensin 3 (hBD3) (3:1)
Example 8I: AMP126+Protegrin-2 (1:3)
Example 8L: AMP1188

### 9A-9L. Preparation of Disinfectant Solutions

Disinfectant solutions (with pH 5.5-6.5) were prepared, having the following composition:

| Ingredients | % concentration |
|---|---|
| water | 98.1425 |
| dihydrated disodium EDTA | 0.1 |
| tris-hydroxymethyl-aminomethane | 0.4 |
| isodecyl alcohol | 0.45 |
| aminoxide | 0.5625 |
| ethoxylated lanolin | 0.2 |
| isopropyl alcohol | 0.125 |
| chlorhexidine digluconate | 0.02 |
| glycerin | 2 |
| propylene glycol | 2 |
| peptide | 0.00005 |

wherein “peptide” is:
Example 9A: AMP2041
Example 9B: AMP72
Example 9C: AMP126
Example 9D: human Beta-defensin 3 (hBD3)
Example 9E: Maximin H1
Example 9F: Dermaseptin
Example 9G: RTD-5
Example 9H: AMP2041+human Beta-defensin 3 (hBD3) (1:2)
Example 9I: AMP72+Maximin H1 (2:1)
Example 9L: AMP51

### 10A-10L. Preparation of Gels

Gels (with pH 6.5-7.5) were prepared, having the following composition:

| Ingredients | % concentration |
|---|---|
| demineralized water | 92.37 |
| natrosol | 1.1 |
| dihydrated disodium EDTA | 0.1 |
| tris-hydroxymethyl-aminomethane | 0.4 |
| ethoxylated lanolin | 0.5 |
| glycerin | 2 |
| polyvinylpyrrolidone | 1.5 |
| sorbitol | 1.43 |
| isodecyl alcohol | 0.5 |
| dye | 0.0015 |
| chlorhexidine digluconate | 0.02 |
| peptide | 0.00005 |

wherein “peptide” is:
Example 10A: AMP2041
Example 10B: AMP72
Example 10C: AMP126
Example 10D: human Beta-defensin 3 (hBD3)
Example 10E: Lactoferrin (f 17-41)
Example 10F: Arenicin-3
Example 10G: RTD-5
Example 10H: AMP2041+human Beta-defensin 3 (hBD3) (1:1)
Example 10I: AMP72+Lactoferrin (f 17-41) (1:1)
Example 10L: AMP289

### 11A-11L. Preparation of Anti-Seborrhoeic Shampoos

Liquid shampoos (with pH 4.0-5.5) were prepared, having the following composition:

| Ingredients | % concentration |
|---|---|
| demineralized water | 36.23 |
| zinc gluconate | 2 |
| dihydrated disodium EDTA | 0.4 |
| tris-hydroxymethyl-aminomethane | 1.6 |
| aminoxide | 27.5 |
| betaine sol. 30% | 18 |
| Cocamide dea | 3.5 |
| salicylic acid | 2 |
| ethoxylated lanolin | 4 |
| fragrance | 0.4 |
| silicone emulsion | 1 |
| silicone emulsion | 0.2 |
| chlorhexidine digluconate | 0.08 |
| lactic acid (as needed) | about 1 |
| sodium chloride (as needed) | about 1.52 |
| peptide | 0.0002 |

wherein “peptide” is:
Example 11A: AMP2041
Example 11B: AMP72
Example 11C: AMP126
Example 11D: AMP16
Example 11E: Protegrin-2
Example 11F: human Beta-defensin 3 (hBD3)
Example 11G: AMP1189
Example 11H: AMP2041+human Beta-defensin 3 (hBD3) (2:1)
Example 11I: AMP126+Protegrin-2 (1:2)
Example 11L: AMP1188

### 12A-12L. Preparation of Anti-Seborrhoeic Shampoos

Liquid shampoos (with pH 4.0-5.5) were prepared, having the following composition:

| Ingredients | % concentration |
|---|---|
| demineralized water | 36.23 |
| zinc gluconate | 2 |
| dihydrated disodium EDTA | 0.4 |
| tris-hydroxymethyl-aminomethane | 1.6 |
| aminoxide | 3 |
| betaine sol. 30% | 2.9 |
| Cocamide dea | 0.9 |
| salicylic acid | 0.6 |
| ethoxylated lanolin | 4 |
| fragrance | 0.4 |
| silicone emulsion | 0.7 |
| silicone emulsion | 0.15 |
| chlorhexidine digluconate | 0.08 |
| lactic acid (as needed) | about 1 |
| sodium chloride (as needed) | about 1.52 |
| peptide | 0.0002 |

wherein “peptide” is:
Example 12A: AMP2041
Example 12B: AMP72
Example 12C: AMP126
Example 12D: AMP16
Example 12E: Protegrin-2
Example 12F: human Beta-defensin 3 (hBD3)
Example 12G: AMP1189
Example 12H: AMP2041+human Beta-defensin 3 (hBD3) (2:1)
Example 12I: AMP126+Protegrin-2 (1:2)
Example 12L: AMP1188

### 13A-13L. Preparation of an Anti-Seborrheic Solutions

Disinfectant solutions (with pH 5.5-6.5) were prepared, having the following composition:

| Ingredients | % concentration |
|---|---|
| demineralized water | 93.22 |
| zinc gluconate | 0.5 |
| dihydrated disodium EDTA | 0.1 |
| tris-hydroxymethyl-aminomethane | 0.4 |
| urea | 0.2 |
| isotridecanol ethoxylate | 1 |
| Cyclosistem d-panthenol | 0.2 |
| aloe glycolic extract | 0.2 |
| ethoxylated lanolin | 1.5 |
| fragrance | 0.4 |
| glycerin | 1.5 |
| chamomile essence | 0.08 |
| chlorhexidine digluconate | 0.02 |
| omega 6 liposome | 1 |
| peptide | 0.0002 |

wherein “peptide” is:
Example 13A: AMP2041
Example 13B: AMP72
Example 13C: AMP126
Example 13D: AMP16
Example 13E: Protegrin-2
Example 13F: human beta-defensin 3 (hBD3)
Example 13G: AMP1189
Example 13H: AMP2041+human Beta-defensin 3 (hBD3) (2:1)
Example 13I: AMP126+Protegrin-2 (1:2)
Example 13L: AMP1188

### 14A-14L. Preparation of Disinfectants for Surfaces and Supports

Products (with pH 8) were prepared, intended for cleaning and disinfection of surfaces and supports of the dairy industry equipment, said products having the following composition:

| Ingredients | % concentration |
|---|---|
| demineralized water | 99.22995 |
| dihydrated disodium EDTA | 0.1 |
| tris-hydroxymethyl-aminomethane | 0.4 |
| isotridecanol ethoxylate | 0.25 |
| chlorhexidine digluconate | 0.02 |
| peptide | 0.00005 |

wherein “peptide” is:
Example 14A: AMP2041
Example 14B: AMP72
Example 14C: AMP126
Example 14D: Temporin A
Example 14E: Melittin
Example 14F: Magainin 2
Example 14G: LL-37
Example 14H: AMP2041+LL-37 (1:1)
Example 14I: AMP72+Melittin (1:1)
Example 14L: AMP944

### 15A-15L. Preparation of Disinfectants for Surfaces and Supports

Products (with pH 8) were prepared, intended for cleaning and disinfection of surfaces and supports of the dairy industry equipment, said products having the following composition:

| Ingredients | % concentration |
|---|---|
| demineralized water | 93.8404 |
| dihydrated disodium EDTA | 0.8 |
| tris-hydroxymethyl-aminomethane | 3.2 |
| isotridecanol ethoxylate | 2 |
| chlorhexidine digluconate | 0.16 |
| peptide | 0.0004 |

wherein “peptide” is:
Example 15A: AMP2041
Example 15B: AMP72
Example 15C: AMP126
Example 15D: Temporin A
Example 15E: Melittin
Example 15F: Magainin 2
Example 15G: LL-37
Example 15H: AMP2041+LL-37 (1:1)
Example 15I: AMP72+Melittin (1:1)
Example 15L: AMP944
   16A-16L. Preparation of a nourishing cleansing veterinary creams

Creams (with pH 4.5-8) were prepared, having the following composition:

| Ingredients | % concentration |
|---|---|
| demineralized water | 68.471 |
| dihydrated disodium EDTA | 0.4 |
| tris-hydroxymethyl-aminomethane | 1.6 |
| diethylene glycol stearate | 12 |
| 30% betaine sol. | 3.1 |
| aminoxide | 15 |
| vegetable squalene | 3 |
| propylene glycol | 5 |
| perfumed essence | 0.4 |
| color | 0.001 |
| chlorhexidine digluconate | 0.08 |
| peptide | 0.0002 |

wherein “peptide” is:
Example 16A: AMP2041
Example 16B: AMP72
Example 16C: AMP126
Example 16D: Lactoferricin B
Example 16E: Protegrin-1
Example 16F: Indolicin
Example 16G: RTD-5
Example 16H: AMP2041+Indolicin (2:1)
Example 16I: AMP126+Protegrin-1 (1:2)
Example 16L: AMP944

### 17A-17L. Preparation of Anti-Seborrhoeic Shampoos/Creams

Viscous shampoos (with pH 4.0-7.5) were prepared, having the following composition:

| Ingredients | % concentration |
|---|---|
| demineralized water | 36.23 |
| zinc gluconate | 2 |
| dihydrated disodium EDTA | 0.4 |
| tris-hydroxymethyl-aminomethane | 1.6 |
| aminoxide | 3 |
| betaine sol. 30% | 2.9 |
| Cocamide dea | 0.9 |
| salicylic acid | 0.6 |
| ethoxylated lanolin | 4 |
| fragrance | 0.4 |
| silicone emulsion | 0.7 |
| silicone emulsion | 0.15 |
| chlorhexidine digluconate | 0.08 |
| lactic acid (as needed) | about 1 |
| diethylene glycol stearate | 12 |
| peptide | 0.0002 |

wherein “peptide” is:
Example 17A: AMP2041
Example 17B: AMP72
Example 17C: AMP126
Example 17D: AMP16
Example 17E: Protegrin-2
Example 17F: human Beta-defensin 3 (hBD3)
Example 17G: AMP1189
Example 17H: AMP2041+human Beta-defensin 3 (hBD3) (2:1)
Example 17I: AMP126+Protegrin-2 (1:2)
Example 17L: AMP1188

The advantages achieved by the composition according to the present invention are apparent from the detailed description and from the Examples above. In particular, said composition allows to take advantage of the surprising synergistic effect resulting from the presence of the at least one peptide, so that it is advantageously possible to benefit from the efficacy of chlorhexidine while minimizing the risk of triggering mechanisms of resistance by the microorganisms concerned. In fact, it has been shown that the compositions of the invention lead to definitely quantitative and satisfactory results of CFU reduction in a very short time and with drastically reduced amounts of chlorhexidine. It is also noted that also peptides can trigger mechanisms of resistance by the microorganisms concerned, however the combination with chlorhexidine also allows the use of drastically and advantageously low amounts of peptides.

1. A composition comprising chlorhexidine or a salt thereof, and at least one peptide, said at least one peptide consisting of 10-50 amino acids, wherein at least two amino acids are basic amino acids selected from Lys, His, Arg, or a combination thereof, and wherein at least 50% of the amino acids are not hydrophobic amino acids.

2. The composition of claim 1, wherein said salt of chlorhexidine is dihydrochloride, diacetate, digluconate or a mixture thereof.

3. The composition of claim 1, wherein chlorhexidine or a salt thereof is in a concentration up to 0.05 g/ml, preferably up to 0.03 g/ml, and said at least one peptide is in a concentration up to 12,5 ug/ml, preferably up to 6 ug/ml.

4. The composition of claim 1, wherein said at least one peptide is a cationic peptide having a sequence A-B-C-D-C′-B′-A′, wherein:
each unit A independently consists of 1-3 amino acids;
each unit B independently consists of a sulfur-containing amino acid;
each unit C independently consists of 5 amino acids selected from both the group (a) of hydrophobic amino acids and the group (b) of basic amino acids or hydrogen bond-forming amino acids;
unit D consists of glycine and a basic amino acid,
wherein:
(i) said hydrophobic amino acids are selected from: Ala, Phe, Ile, Leu, Pro, Tyr, Trp and Val;
(ii) said basic amino acids are selected from: Lys, His, Arg;
(iii) said hydrogen bond-forming amino acids are selected from Asn, Gln, Ser, Thr;
and where the substructure C-D-C′ contains a total of 5 to 9 points of alternation between an amino acid of group (a) and an amino acid of group (b) or vice versa.

5. The composition of claim 4, wherein the peptide is in a cyclized form by formation of a disulfide bridge between the two units B.

6. The composition of claim 4, wherein each unit A and A′ of the peptide independently consists of 1 or 2 amino acids and at least one of the units C and C′ comprises Lys.

7. The composition of claim 1, wherein both units C and C′ of the peptide comprise Lys.

8. The composition of claim 1, wherein the unit D of the peptide is -Arg-Gly-.

9. The composition of claim 8, wherein the amino acid adjacent to Gly of unit D of the peptide is a hydrophobic amino acid, preferably an aromatic hydrophobic amino acid.

10. The composition of claim 1, wherein the sequence of the peptide consists of 17 amino acids, wherein the units A and A′ independently consist of 1 or 2 amino acids, the units B and B′ are both Cys, at least one of the units C and C′ comprises Lys, all the amino acids in position 6, 8, 13 (numbered from A to A′) belong to said group (i) of hydrophobic amino acids, and amino acids in position 9 and 10 are Arg and Gly.

11. The composition of claim 1, further comprising a buffer solution comprising a buffer compound selected from TRIS (or tris(hydroxymethyl)aminomethane), PIPES (or piperazin-1,4-bis (2-ethanesulfonate acid)), HEPES (or 4-2-hydroxyethyl-1-piperazinyl-ethanesulfonic acid), sodium phosphate monobasic and dibasic acid, or citric acid, and comprising a sequestering agent selected from EGTA (ethyleneglycoltetraacetic acid), EDTA (ethylenediaminetetraacetic acid) or an anhydrous or hydrated-salt form thereof, calcium disodium EDTA or a hydrated form thereof, diammonium EDTA or a hydrated form thereof, dipotassium EDTA or a hydrated form thereof, disodium EDTA or a hydrated or dihydrated form thereof, TEA-EDTA (EDTA salt of mono (triethanolamine))tetrasodium EDTA, tripotassium EDTA, trisodium EDTA, HEDTA (hydroxyethyl-ethylenediaminotriacetic acid), HEDTA-EDTA, and mixtures thereof.

12. The composition of claim 11, wherein said buffer compound is in a concentration up to 1 g/ml and said sequestering agent is in a concentration up to 0.5 g/ml.

13. The composition of claim 12, comprising up to 0.0025 g/ml of chlorhexidine or a salt thereof, up to 10.0 ug/ml of at least one peptide, up to 0.5 g/ml of buffer compound, and up to 0.2 g/ml of sequestering agent.

14. The composition of claim 13, comprising up to 0.002 g/ml of chlorhexidine or a salt thereof, up to 5.0 ug/ml of at least one peptide selected from SEQ.ID.No. 1-29 and 31-54, up to 0.1 g/ml of buffer compound, and up to 0.01 g/ml of sequestering agent.

15. The composition of claim 14, wherein said at least one peptide is selected from SEQ.ID.No.1, SEQ.ID.No.2, and SEQ.ID.No.3.

16. The composition of claim 11, wherein said buffer solution comprises TRIS and EDTA disodium dihydrate.

17.-23. (canceled)

24. The composition of claim 1, in the form of aqueous solution, anhydrous solution, dispersion, emulsion, suspension, liniment, cream, paste, gel, ointment, shampoo, powder, aerosol, soft or hard capsule, tablet, mini-tablet, micro-tablet, granule, micro-granule, pellet, multiparticulate, micronized particles, pill, syrup, oil, lotion, drops, eye drops, liposomes, nanoparticles, patches, bandages and dressings.

25. A method of treatment of infections caused by bacteria, fungi and/or yeasts, comprising the step of administering an effective amount of the composition of claim 1 to a subject in need thereof.

26. The method of claim 25, wherein the composition is in a topically administrable form.

27. A method for the disinfection and sanitization of surfaces or supports, comprising the step of applying a composition of claim 1 to said surfaces or supports.
